(19) United States
(12) Patent Application Publication
(10) Pub. No.: US 2006/0052726 A1
(43) Pub. Date: Mar. 9, 2006

(54) Key device to measure pronation and supination of the forearm

(76) Inventors: Donald J. Weisz, New York, NY (US); Jean-Michel Gracies, New York, NY (US)

Correspondence Address: DARBY & DARBY P.C., P. O. BOX 5257, NEW YORK, NY, 10150-5257, US

(21) Appl. No.: 10/937,517

(22) Filed: Sep. 8, 2004

(51) Int. Cl. A61B 5/103

(52) U.S. Cl. 600/595

(57) ABSTRACT

There is disclosed herein a method for quantifying a user's motor skills. The method comprises the steps of voluntarily moving a motion sensing member for at least a part of one cycle, the cycle comprising movement in a first direction and a return direction, measuring at least one of regularity of the movement of the member and mean angular velocity of the member. The method may be performed simultaneously with two members. Further, the members may be actuated by various body parts including a finger, a hand, or a shoulder. The data generated by the movement of the motion sensor may be stored as modulated carrier frequency in order to perform the method in locations remote from laboratories or computing facilities.

### FIELD OF THE INVENTION

This invention relates to a method for quantifying motor skills of the shoulders, wrists, hands, and fingers in order to diagnose more effectively diseases affecting movements and the degree of disease progression.

### BACKGROUND OF THE INVENTION

Human movements can be categorized by speed into slow, ballistic, and rapid. In slow movement, a conscious effort is made to minimize the displacement speed of the body segment. The ability to perform slow movements is important to tasks of accuracy, e.g., drawing complex geometric figures. In ballistic movement, a conscious effort is made to maximize displacement speed and acceleration. Ballistic movements might be used in situations of emergency, or of physical competition (sports). Slow and ballistic movements are usually discrete, i.e., the limb segment does not return to its initial position following the same course and at the same speed as in the initial movement. The third movement type is rapid movement, in which speed is neither minimized nor maximized consciously. Most purposeful movements in everyday life are rapid, e.g., in walking, reaching, writing, etc. Most rapid movements are not discrete but alternating. In alternating movements a body segment moves in one direction and returns to the initial position following a similar course in space and at similar speed (e.g., leg movements when walking, wrist movements when writing, shoulder and elbow movements when reaching and retrieving, finger movements when grasping and releasing, jaw movements when chewing, etc.). Rapid Alternating Movements (“RAMs”) are thus essential in daily functioning, and RAM disturbances, referred to as dysdiadochokinesia, may significantly impact on activities of daily living.

The present invention is based on the observation that the most common neurological disorders of movement, such as Parkinson's disease (PD), significantly impair RAM; consequently, tests of RAM are a standard and critical assessment in the clinical setting. Clinically, some disorders (e.g. Parkinson's disease) affect large movements more than small movements, whereas others (e.g. cerebellar dysfunction) appear to affect small movements more than large ones.

The present invention has been used to test the effectiveness of deep brain stimulation in PD patients who have had electrical stimulators implanted into the subthalamic nucleus. These deep brain stimulators (DBS) are programmed to stimulate a discrete brain area with a low voltage at greater than 100 Hz. Although in most patients DBS leads to a reduction in the severity of symptoms and/or a reduction in the dosage of adjuvant medication needed, there is a wide range in the effectiveness across implanted patients. The present invention is able to measure accurately the effects of DBS by means of at least two measurements: maximum velocity achieved during pronation-supination cycles and regularity of movement.

Currently, devices that can quantify RAMs in individuals with movement disorder have been used in laboratory settings, but none of those tests can easily be adapted to clinical examination because of their lack of portability. In the clinical setting, none of the tests currently available is capable of precisely quantifying rapidly alternating movements. This situation hinders assessment of disorder type and progression and selection and adjustment of therapy.

Thus, there is a need for a method and device that may be easily used in a variety of clinical settings in order to quantitatively assess rapid alternating movement performed by patients who are affected by motor disorders. The invention addresses these and other needs in the art.

### SUMMARY OF THE INVENTION

The present invention provides a method for quantifying a user's motor skills. A standardized task is performed by the user, the task comprising successive cycles of alternating movements of a member. Multiple characteristics of the movement (e.g. mean angular velocity, maximum angular velocity, acceleration, jerk, smoothness, and regularity) are measured to provide a quantitative indication of the user's motor skills.

Further, the invention provides a method for quantifying movements of any size (large or small), whether pre-specified or not. Examples of movements involving pre-specified sizes include those made to a pre-defined target or those made when physical blocks are placed at pre-defined points. The present invention also provides a method for measuring only the clockwise, or in the alternative, only the counter-clockwise portions of the cycles of clockwise-counterclockwise rapidly alternating movements.

Finally, the present invention provides for measuring the simultaneous bimanual operation of two devices, each capable of measuring multiple movement characteristics (e.g. mean angular velocity, maximum velocity, acceleration, jerk, smoothness, and regularity).

### BRIEF DESCRIPTION OF THE DRAWINGS

The above features and many attendant advantages of the invention will be better understood by reference to the following detailed description when taken in conjunction with the accompanying drawings, in which:

FIG. 1 is a perspective view from the front of a preferred embodiment of the device used to measure pronation and supination of the forearm;

FIG. 2 is a perspective view from the rear of the embodiment shown in FIG. 1.

FIG. 3 illustrates a set of traces representing a 10-second sample of the output of the device in which the movement of a patient's forearm is measured. In addition the figure shows the results of typical manipulations of the output. From the bottom to the top, the traces show the output of the device in volts, the output after conversion to degrees, velocity (degrees/sec), acceleration (degrees/sec²), jerk (degrees/sec³), and rectified jerk.

FIG. 4 is a chart showing the mean percent change in the maximum angular velocity attained by five patients with Deep Brain Stimulators active.

FIG. 5 is a chart showing the percent change in maximum angular velocity for five patients with Deep Brain Stimulators.

FIG. 6 is a chart showing individual results for five patients comparing the movement variability when the Deep Brain Stimulators are on versus when they are off.

FIG. 7 is a chart showing a comparison of mean angular velocity of forearm pronation-supination in age-matched control subjects and five patients with Parkinson's disease with Deep Brain Stimulation both on and off.

FIG. 8 is a chart showing the variability of pronation-supination movement in successive cycles. The three sets of data correspond to Parkinson's patients with Deep Brain Stimulators on, with those stimulators off, and age matched controls.

FIG. 9 is a chart showing the mean angular velocity of forearm pronation-supination in a Parkinson's patient with a therapeutic dose of L-DOPA versus the same patient without the dose of L-DOPA.

FIG. 10 is a chart showing a comparison in the variability of a Parkinson's patient's movement in successive cycles of forearm pronation-supination, with one set of data generated when the patient has been given a therapeutic dose of L-DOPA versus the same patient without the dose of L-DOPA.

FIG. 11 is a perspective view showing another embodiment of the device, which incorporates shoulder and elbow tasks.

FIG. 12 is an enlarged view of the embodiment illustrated in FIG. 11 with the U-shaped clamp.

FIG. 13 perspective view of another embodiment of the device, which incorporates extension and flexion of a finger.

### DETAILED DESCRIPTION OF THE INVENTION

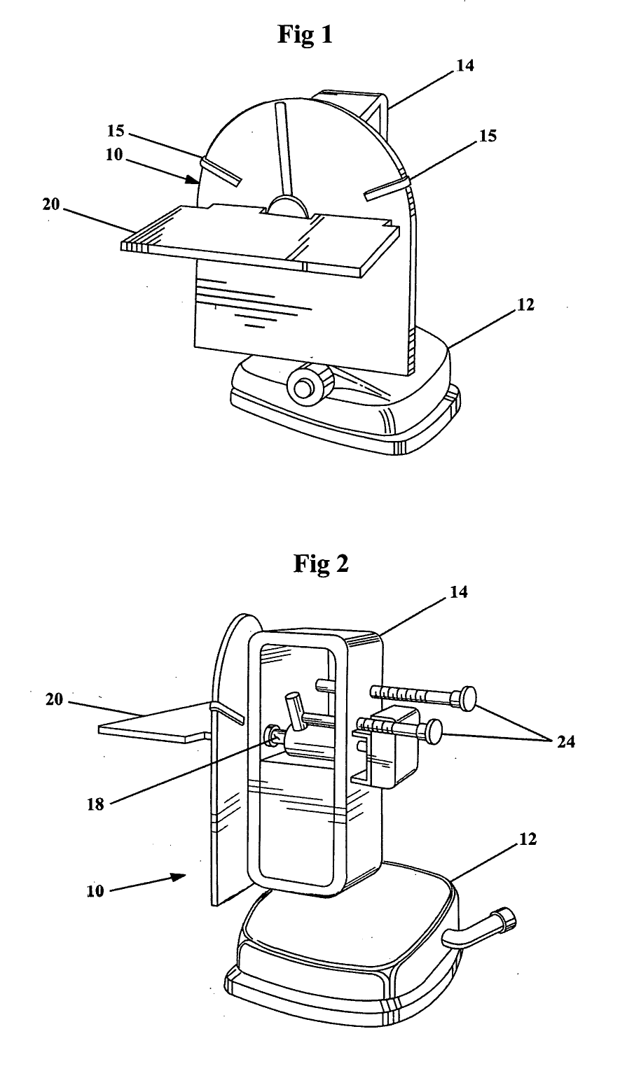

Referring to FIG. 1, the preferred embodiment of the device to measure pronation and supination of the forearm is shown. The subject is directed to alternate between moving a member 20 clockwise (“CW”) and counter-clockwise (“CCW”). The position of the member 20 is continuously measured and recorded. Calculations are performed in order to quantify the subject's movement.

The construction of the device used to gather data describing RAM and the method for using the data to precisely quantify the progression of a disease affecting motor skills is described by reference to the device designed to measure the CW-CCW cycles of a forearm (also know as the pronation-supination cycles). However, the same measurements can be performed with the finger or shoulder/elbow by modifying the forearm device as described herein.

In the exemplary embodiment of the present invention, the device 10 is mounted to a base 12 that permits the device to be firmly attached to a smooth, flat surface or alternatively to the edge of a flat surface. A box 14 is secured to the base 12 by conventional means. At the rear of the box 14 is an optical encoder that converts the rotary position of the shaft 18 to a voltage. Thus, the actuating member in combination with the optical encoder constitutes a motion sensing member. Affixed to the front of the box, and connected to the shaft, is an actuating member 20, which is sized to accommodate the palm of a hand. The position of screws 24, mounted on the inner wall, determine the maximal movement of the actuating member 20 in the clockwise and counterclockwise directions.

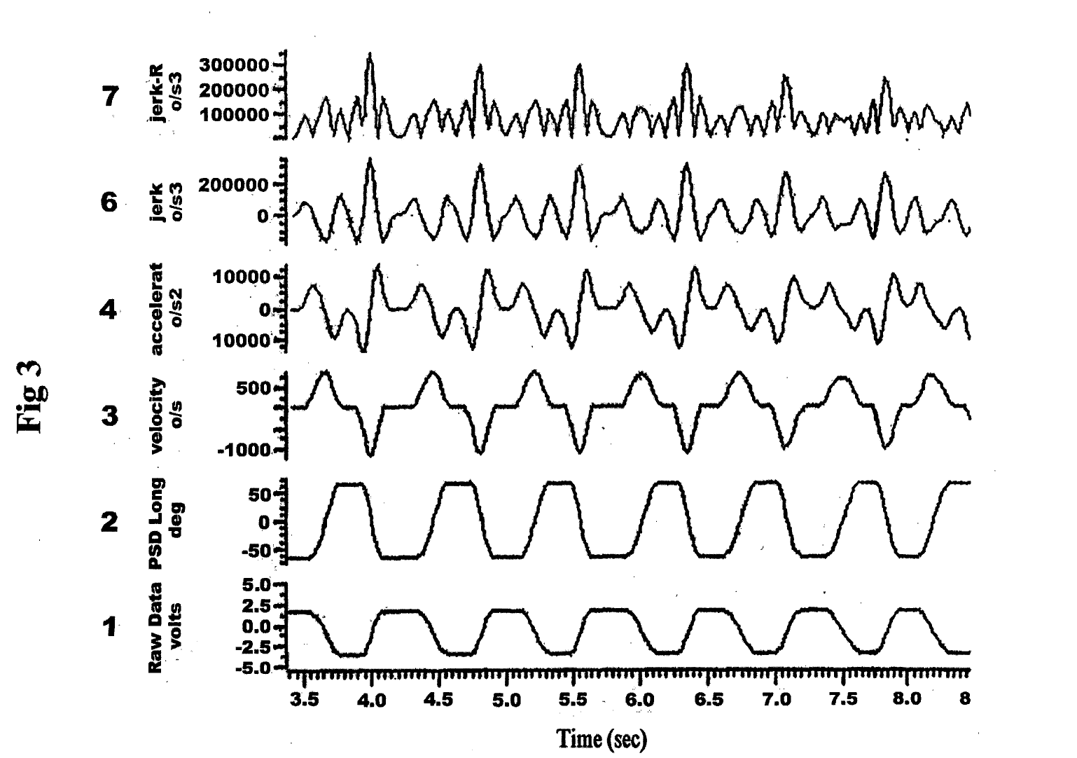

The user of the device is instructed to engage the member 20 while performing RAMs. In one exemplary embodiment, the user may be instructed to make only small alternating movements (e.g., 45°). In another exemplary embodiment the user is instructed to make only large alternating movements (e.g., 135°). Sliding bars when locked into place restrict movement and thereby determine maximal movement size. In one exemplary embodiment two movement-limiting bars can be placed at 45° apart or alternatively at 135° apart. In another embodiment, targets that do not restrict movement can also be employed if desired. In this embodiment subjects are instructed to move the actuating member 20 until a pointer that is attached is pointing at the target 15 in the clockwise direction. The subject then moves until the actuating member 20 until the pointer is aligned with the second target 15 placed in the counterclockwise direction. During any movements the user makes, the angular position of the actuating member 20 is continuously monitored and recorded. From this data, the mean angular velocity, maximum velocity, acceleration, jerk, smoothness, and variability may be calculated. A sample 10-second output of the device is shown in FIG. 3.

The data created by the movements of the actuating member 20 can be recorded in two ways. One is to connect the optical encoder of the device directly to a computer that has appropriate converters and data acquisition software. The second is to use the output of the optical encoder to modulate a carrier frequency that is generated by a simple electronic circuit that can be entirely contained within the device. The carrier frequency can then be saved as a file on a small voice recorder or an MP3 player/recorder. Thus, by using the second method of recording the data created by the movements of the actuating member, the device is rendered portable.

One example of possible tasks to be measured includes securing a device to a smooth, hard surface at a distance that depends on the length of the subject's forearm. The subject is seated at a table and the subject's forearm is placed wholly on the table. The point of the elbow (specifically the olecranon) is placed in a rubber pad that has a hole in the center. The subject grasps the actuating member 20 by placing only the thumb on top of the member 20 and the elbow pad is placed at a distance that permits easy rotation of the member 20 while the subject maintains a straight wrist. The subject can then perform one of several tasks usually for a period of 15 sec.

FIG. 3 shows a sample of the plots from the output of the optical encoder in the device of the exemplary embodiment. From the bottom up, the channels are the raw data (in volts), the position in degrees with 0° being when the member 20 is parallel to the table, then the velocity of the movement, the acceleration, the jerk (the third derivative of position), and rectified jerk. The rectified jerk is being used as an additional measure of the smoothness of the movements. The mean rectified jerk increases as the smoothness of the movement decreases.

Mean angular velocity generated by a user with no motor skill impairment will be higher than that generated by a patient with a disorder affecting motor skills. The present invention is able to quantify accurately the mean angular velocity of the user's motion, thus enabling comparison on a more precise level. The present invention has been used to quantify the effects of Deep Brain Stimulation (DBS) therapy on patients with motor skill disorders caused by Parkinson's disease. The result of the study showed a measurable increase in the mean angular velocity of motion produced by patients when the DBS was turned on versus when it was turned off.

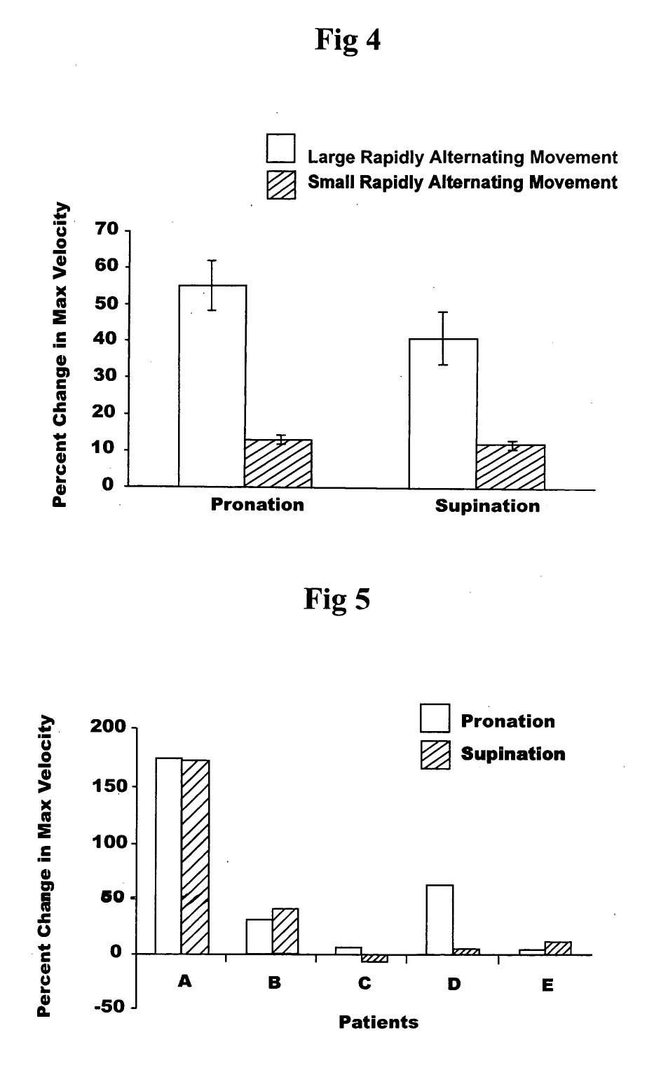

FIG. 4 shows the mean percent change in the maximum velocity attained by PD patients with their stimulators ON relative to the OFF condition. Values for pronation are plotted separately from the values for supination for five PD patients. As is seen in FIG. 4, the ON condition produced improvements in both small and large movements, however, the large movements showed greater improvement than did the small movements. FIG. 5 shows the individual data for the same five PD patients focusing on large movements. The high variability in the effectiveness of DBS in these five patients can be seen in the values for maximum velocity. Patient A showed a greater than 150% increase in maximum velocity whereas Patients C and E showed very little change from DBS ON to the OFF conditions. These results parallel other clinical observations of the effects of DBS on Patients C and E. These two patients benefited less from the DBS than did the other patients.

Maximum velocity is only one measure of movement. The present invention can employ any or all of several measures for detecting differences in the movements produced using the device. For one of these measures, variability, each complete cycle of pronation and supination is divided into twelve equal segments. The twelve segments, defined for each complete cycle of supination and pronation by 13 equally-spaced time points, are based on the time it took to complete each specific cycle. If a subject performs consistently and smoothly, then the positions of the member 20 at each of the time points should be very similar across all of the cycles. If, however, the movement has a high number of accelerations and decelerations, then the positions of the member 20 at each of those equal time points will vary. This is true even if the overall velocity of the movements varies because of the normalization process that is performed when each cycle of pronation and supination is divided into the twelve equal segments. For this analysis, the first and last time points are discarded because they are by definition invariant. The position of the member 20 (in degrees) at each of the remaining 11 time points is used to calculate the amount of variability that the subject exhibits across all of the movements for each task. One of the major symptoms of Parkinson's disease is the difficulty to produce smooth movements, especially large movements like taking a step or moving an arm in a wide arc. One prediction would be that variability in the shapes of the forearm pronation-supination movements should decrease if DBS were effectively improving movement in individuals with Parkinson's disease.

The mean and the standard deviation of angular displacement at each of the 11 time points are calculated and the standard deviation of angular displacement across all cycles is determined. The standard error of angular displacement is then calculated. By using standard error, the variability measure is normalized for the number of complete cycles performed within 15 seconds. This measure then represents the variability of pronation-supination cycles across movements performed by the same subject while eliminating any bias due to velocity. A very low variability score would indicate that the topographies of all pronation-supination movements were very similar even if the speed varied.

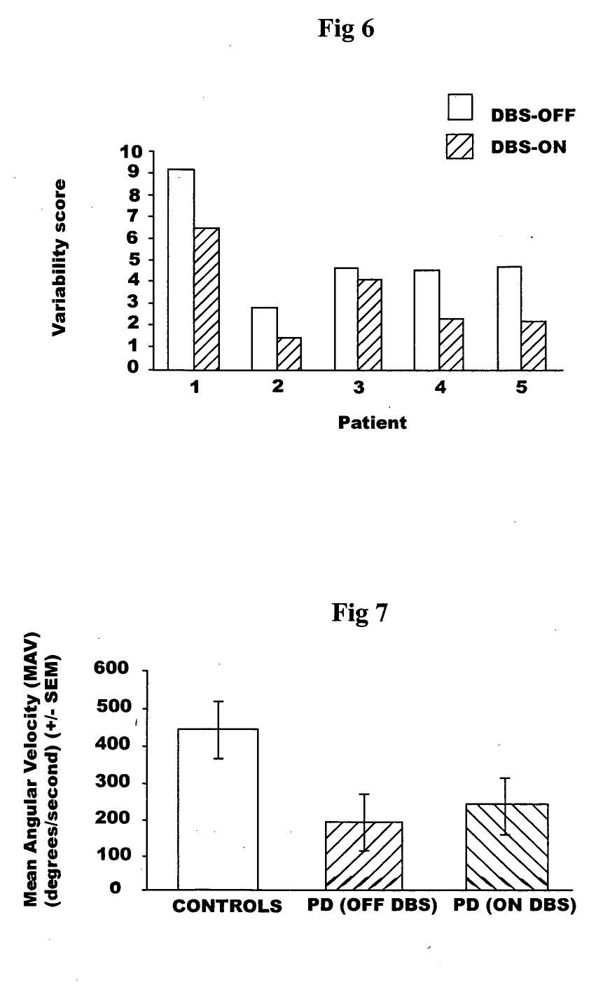

FIG. 6 shows the percent variability when DBS is ON versus OFF in the same five DBS patients. In all five patients the variability in the shapes of the pronation-supination movements was reduced when the DBS was ON relative to the OFF condition. For this test, the reduction in variability across cycles of pronation and supination with DBS ON relative to OFF was similar for the large and small movements.

FIG. 7 depicts the mean angular velocity for five patients with Parkinson's disease who had previously undergone surgery for implantation of a deep brain stimulator targeted for the subthalamic nucleus. Parkinson's patients were tested in two conditions, stimulator off (PD OFF DBS) and stimulator on (PD ON DBS). The PD patients performed movements much more slowly than did age-matched controls. In PD patients, there was an increase in mean angular velocity in the ON DBS condition although the increase was small (24.4% increase) relative to the much larger difference between controls and PD patients.

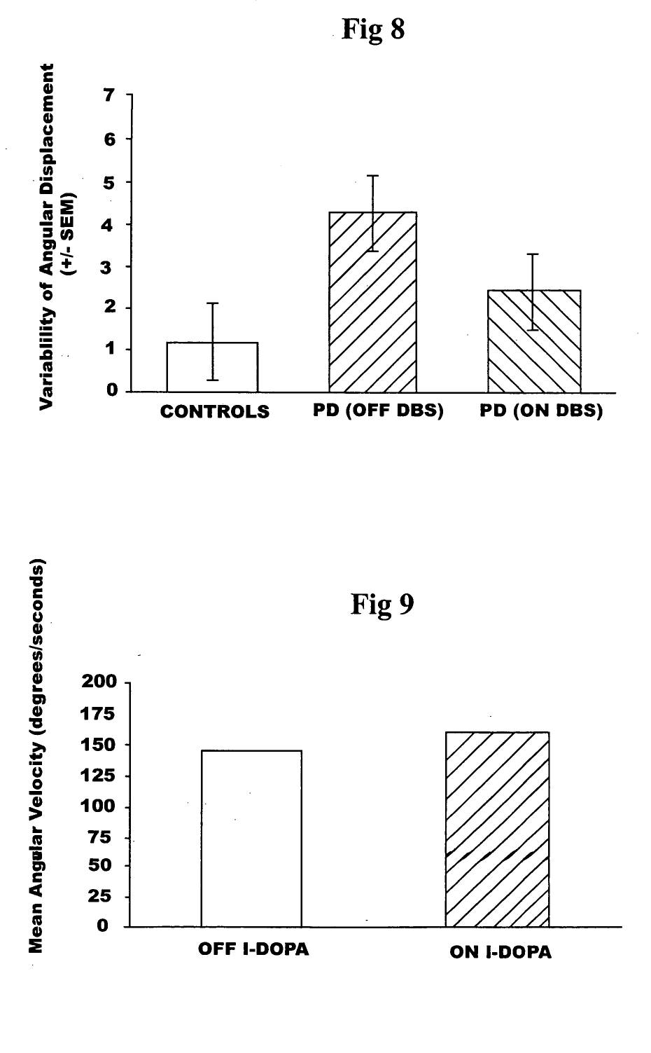

FIG. 8 shows the extent to which the topographies of successive forearm pronation-supination cycles varied across testing. Age-matched controls had the lowest level of variability on this measure. The variability of successive forearm movements in PD patients improved markedly when their stimulators were turned on. The standard error measure of variability of movement decreased by approximately 42% from the DBS OFF condition to the DBS ON condition.

The effects of a therapeutic dose of L-DOPA on mean angular velocity and variability of successive cycles of forearm pronation-supination movements were evaluated in one PD patient. FIG. 9 shows that in the patient, L-DOPA produced only a small effect on velocity.

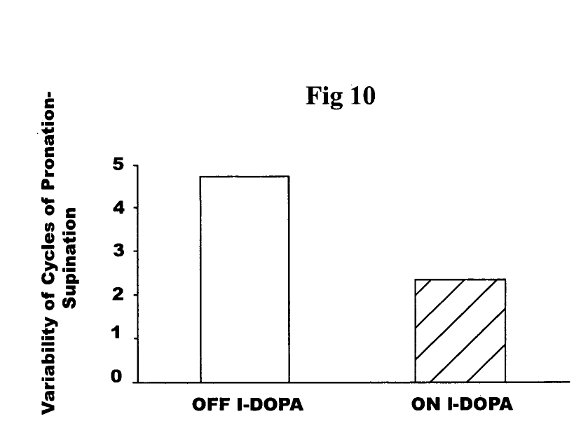

On the other hand, FIG. 10 shows that the drug markedly reduced the variability of successive cycles of pronation-supination in the same patient. Detection of this type of variability requires a tool capable of monitoring the entire movement, as the present invention can.

The direct comparison between large (e.g., 135°) and small (e.g., 35°) alternating movement measures the capacity to scale movement, independently from the capacity to change movement direction. Such measures may assist diagnosis, as large movements are more affected than small movements in motor disorders such as Parkinson's disease, while it is the opposite in other (e.g. frontal or cerebellar).

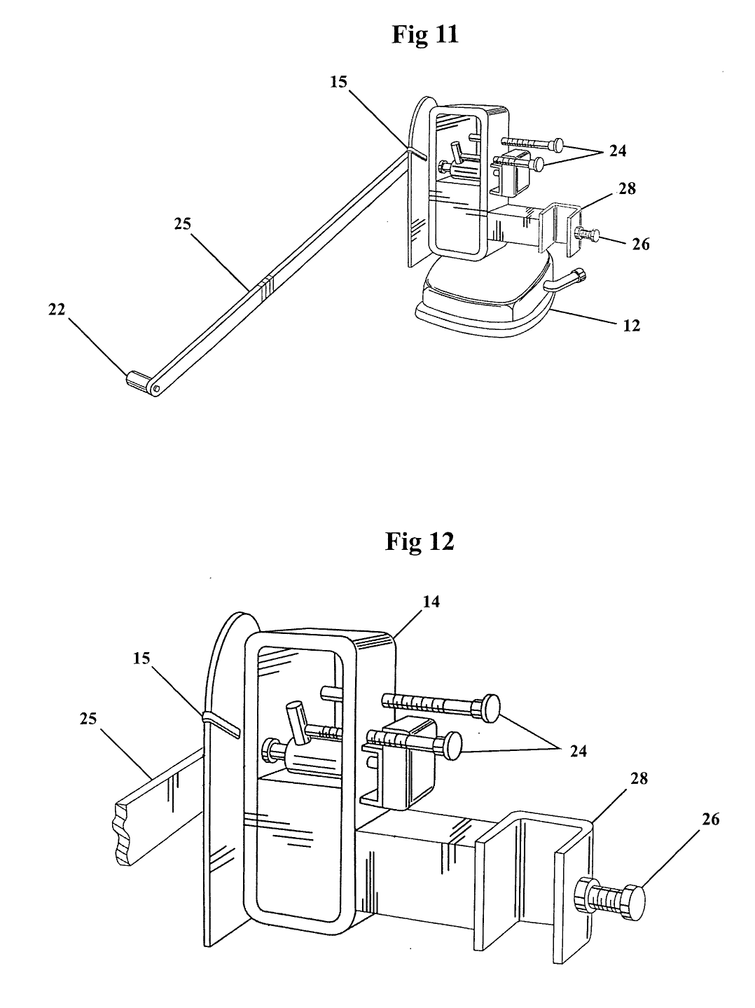

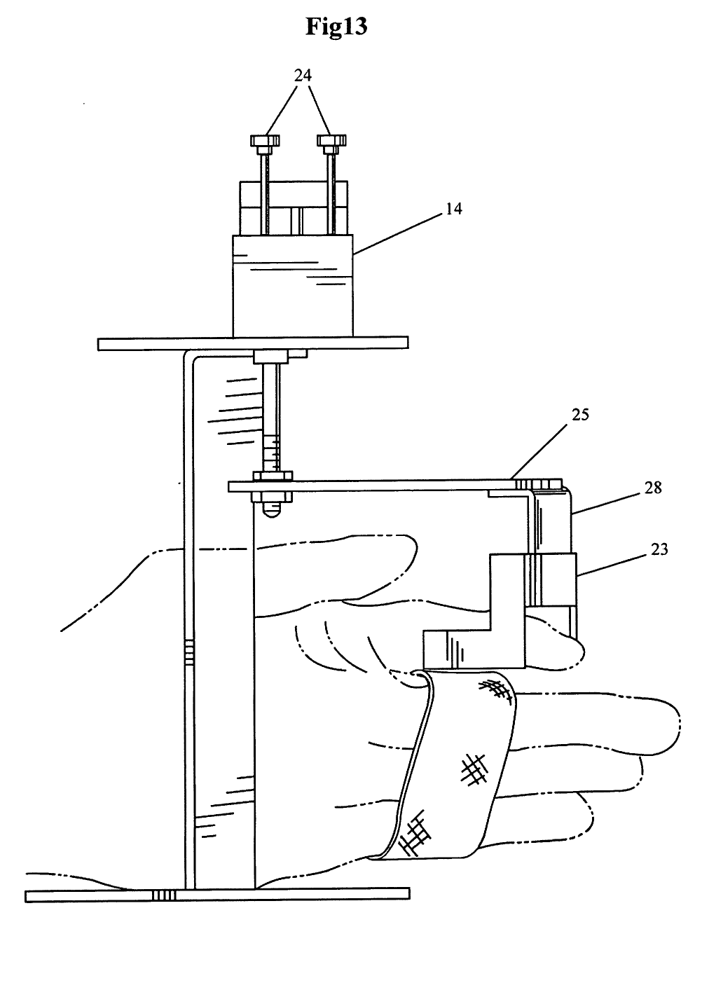

In an alternate embodiment of the present invention shown in FIG. 11 circular motion that is produced by either shoulder muscles or, alternatively, elbow muscles can be measured. Two steps are taken to convert the device to one that measures shoulder or elbow motion. First, the member 20 is removed by loosening a set screw and a crank 25 is inserted in its place and secured to the rotary shaft by re-tightening the set screw. Second, a U-shaped clamp 28 at the rear of the device is mounted at the edge of an open door by inserting the edge of the door into the clamp and tightening a set screw 26 on the opposite side of the door. The door is then closed prior to use. In this embodiment the subject grasps the handle 22 of the crank 25 and moves it in a circular motion using only shoulder muscles or in another set of tasks only elbow muscles. This use of the elbow or shoulder motion in CW-CCW cycles is often referred to as rotation cycles. All of the measurements described for the device actuated by the forearm can be applied to movements made when subjects turn the crank handle 22.

In another embodiment shown in FIG. 13, the member 20 (or crank 22) is removed from the rotary shaft and a finger holster 23 is attached to the shaft. The device is then mounted and secured at the top of a U-shaped bracket with the shaft of the device pointing straight down. The hand is placed under the device and the index finger is secured in the holster 23. The device in this embodiment continuously records the position (i.e. angle) of the index finger of either one and/or both hands, while the subject performs finger taps, i.e., the subject would be instructed to place his finger in the holster and then move the finger back and forth. This motion by the finger is often described as extension-flexion motion. Finger tap is used universally in the assessment of both psychological and motor function. The fingers, compared with all other limb segments in the body, have the largest representation in the motor cortex relative to their size. Therefore, it is particularly valuable to be able to quantify finger movements in a patient assessed for motor function. All of the measurements described for the device actuated by the forearm can be applied to the movements made when subjects move their index fingers.

Any of the devices described herein may be operated bimanually. For example, in the exemplary embodiment, two separate devices 10, one for each forearm, may be rotated at the same time. Recently it has been shown that bimanual tasks in which the two forearms are performing opposite phases of a pronation-supination task (e.g. supination in one and concurrently pronation in the other) selectively increase activity in some motor areas of the brain compared to unimanual tasks.

Finally, the present invention includes measuring and comparing the CW segments of CW-CCW cycles with CCW segments of the same cycles. This comparison can be seen in the individual plots of pronation and supination in FIG. 3. The clinical value of this measure is still being assessed.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

What is claimed as new and desired to be protected by Letters Patent of the United States is:

1. A method for quantifying a user's motor skills, comprising the steps of:
voluntarily moving a motion sensing member for at least a part of one cycle, the cycle comprising movement in a first direction and a return direction;
measuring at least one of regularity of the movement of the member and
mean angular velocity of the member.

2. The method of claim 1, wherein data generated by the motion sensing member is saved as a modulated carrier frequency.

3. The method of claim 1, wherein the measuring step comprises calculating the mean angular velocity for one cycle.

4. The method of claim 1, wherein the measuring step comprises calculating the mean angular velocity during movement in the first direction.

5. The method of claim 1, wherein the measuring step comprises calculating the mean angular velocity during movement in the return direction.

6. The method of claim 1, wherein the measuring step comprises calculating the regularity for one cycle.

7. The method of claim 1, wherein the measuring step comprises calculating the regularity during movement in the first direction.

8. The method of claim 1, wherein the measuring step comprises calculating the regularity during movement in the return direction.

9. The method of claim 1, wherein the measuring step comprises calculating the mean angular velocity for movements between 30 and 60 degrees.

10. The method of claim 1, wherein the measuring step comprises calculating the mean angular velocity for movements between 110 and 150 degrees.

11. The method of claim 1, wherein the measuring step comprises calculating the regularity for movements between 30 and 60 degrees.

12. The method of claim 1, wherein the measuring step comprises calculating the regularity for movements between 110 and 150 degrees.

13. The method of claim 1, wherein the motion sensing member is constructed to be actuated about an axis along the length of the arm.

14. The method of claim 1, wherein the motion sensing member is constructed to be actuated by pivoting an arm at a shoulder.

15. The method of claim 1, wherein the motion sensing member is constructed to be actuated by pivoting an index finger through the center of the finger.

16. A method for quantifying a user's motor skills, comprising the steps of:
moving two members bimanually in a first direction and in a return direction;
measuring at least one of regularity and mean angular velocity of each member for at least part of one cycle.

17. A method for diagnosing and detecting neurological disorders effecting motor skills, comprising the steps of:
detecting the voluntary movement of a portion of the body through at least a portion of a cycle comprising consecutive movements in opposite directions, and
measuring one of the regularity of the movement and the mean angular velocity of the movement.

18. The method of claim 17, wherein the disorder is Parkinson's Disease.

19. The method of claim 17, wherein the portion of the body is a forearm.

20. The method of claim 17, wherein the portion of the body is a finger.

21. The method of claim 17, wherein the portion of the body is an arm.
